(19) United States
(12) Patent Application Publication
(10) Pub. No.: US 2009/0053301 A1
(43) Pub. Date: Feb. 26, 2009

(54) SILICONE VESICLES CONTAINING ACTIVES

(76) Inventors: Shaow Lin, Midland, MI (US); James Thompson, Sanford, MI (US)

Correspondence Address: DOW CORNING CORPORATION CO1232, 2200 W. SALZBURG ROAD, P.O. BOX 994, MIDLAND, MI, 48686-0994, US

(21) Appl. No.: 12/279,352

(22) PCT Filed: Jan. 12, 2007
(86) PCT No.: PCT/US07/00833

§ 371 Date: Aug. 14, 2008

Related U.S. Application Data
(60) Provisional application No. 60/777,665, filed on Feb. 28, 2006.

(51) Int. Cl. A61K 9/127; A61Q 17/04; A61P 17/00

(52) U.S. Cl. 424/450; 424/59

(57) ABSTRACT

A process is disclosed for preparing a hydrophobic active loaded vesicle composition by admixing a hydrophobic active to a pre-formed silicone vesicle dispersion. The silicone vesicle compositions are useful in a variety of personal and healthcare compositions.

### CROSS REFERENCE TO RELATED APPLICATIONS

This application claims the benefit of U.S. Provisional Patent Application Ser. No. 60/777,665, filed on 28 Feb. 2006, under 35 U.S.C. §119(e). U.S. Provisional Patent Application Serial No. 60/777,665 is hereby incorporated by reference.

### TECHNICAL FIELD

This invention relates to a process for preparing a hydrophobic active loaded vesicle composition by admixing a hydrophobic active to a pre-formed silicone vesicle dispersion. The present invention also relates to the vesicle compositions prepared according the present process, as well as personal care compositions containing the silicone vesicle compositions.

### BACKGROUND

WO 2005/103157 discloses a process for preparing silicone vesicles from an organopolysiloxane having at least one hydrophilic substituent group by dispersing the organopolysiloxane in a water miscible volatile solvent, with water to form an aqueous dispersion, and then removing the water miscible volatile solvent to form the vesicles in aqueous continuous phase. These type of vesicles may be described as “assembly-required vesicles”, as usually the organopolysiloxane used to make them are hydrophobic and do not spontaneously form vesicles upon dispersion in water.

WO 2005/102248 describes a process for preparing an active containing vesicle composition comprising: I) combining A) an organopolysiloxane having at least one hydrophilic substituent group, B) a water miscible volatile solvent, C) optionally, a silicone or organic oil, D) a personal care or health care active with water to form an aqueous dispersion, II) mixing the aqueous dispersion to form vesicles, and III) optionally, removing the water miscible volatile solvent from the vesicles. Actives are incorporated into assembly-required vesicles following this method. However, it is necessary that actives be incorporated into the step (I) of the process. This may limit the utility of these vesicles since the actives must be incorporated at the vesicle-forming stage of the process. Thus, there is a need for a method to incorporate various actives into the aforementioned silicone vesicles without requiring the actives be present during the vesicle formation step.

The present inventors have unexpectedly discovered that various actives can be incorporated or entrapped within the aforementioned silicone vesicles compositions by “post addition” of the actives to the formed silicone vesicles. In particular, hydrophobic actives may be post added to the aforementioned silicone vesicles and further mixed to yield stable vesicle compositions in which the hydrophobic active is entrapped within the silicone vesicle.

### SUMMARY

This invention provides a process for preparing a hydrophobic active loaded vesicle composition comprising:
I) combining;
   A) an organopolysiloxane having at least one hydrophilic substituent group,
   B) a water miscible volatile solvent,
   C) optionally, a silicone or organic oil, with water to form an aqueous dispersion,
II) mixing the aqueous dispersion to form a vesicle dispersion,
III) optionally, removing the water miscible volatile solvent from the vesicle dispersion, and then
IV) admixing to the vesicle dispersion;
   D) a hydrophobic active to form the hydrophobic active loaded vesicle composition.
      The present invention also relates to the vesicle compositions prepared according the present process, as well as personal care compositions containing these vesicle compositions.

### DETAILED DESCRIPTION

Step I) of the process of the present invention involves combining;
A) an organopolysiloxane having at least one hydrophilic substituent group,
B) a water miscible volatile solvent,
C) optionally, a silicone or organic oil.
   with water to form an aqueous dispersion. Components A)-C) are described below.

### A) Organopolysiloxane Component

Component A) is an organopolysiloxane having at least one hydrophilic substituent group. Organopolysiloxanes are well known in the art and are often designated as comprising any number of “M” siloxy units (R₃SiO_{0.5}), “D” siloxy units (R₂SiO), “T” siloxy units (RSi_{1.5}), or “Q” siloxy units (SiO₂) where R is independently any hydrocarbon group. In the present invention, the organopolysiloxane has at least hydrophilic substituent. That is, at least one of the R hydrocarbon groups present in the organopolysiloxane is a hydrophilic group. For purposes of this invention, “hydrophilic group” is the accepted meaning in the art, i.e. designating water loving chemical moieties. Thus, the hydrophilic group can be selected from various cationic, anionic, zwitterionic, polyoxyalkylene, oxoazoline chemical moieties that are commonly used in combination with various hydrophobic chemical moieties to create surfactant structures or molecules having surface-active behavior.

The amount of the hydrophilic substituent on the organopolysiloxane can vary, depending on the specific chemical component, providing there is at least one hydrophilic group present on the organopolysiloxane. However, the amount of the hydrophilic groups present in the organopolysiloxane can be described by its weight percent, or in particular, the weight percent of the organopolysiloxane and weight percent of the total hydrophilic groups present in the molecule. Typically, the weight percent of the siloxane units in the organopolysiloxane can vary from 20 to 85, alternatively from 30 to 85, or alternatively from 35 to 80 weight percent, while the remaining weight portion of the organopolysiloxane is the hydrophilic group.

In one embodiment of the present invention, the organopolysiloxane having at least one hydrophilic substituent group is selected from silicone polyethers. Silicone polyethers (SPEs) generally refer to silicones containing polyether or polyoxyalkylene groups, which could take in many different structural forms. One such form is rake-type SPEs which are derived most commonly from hydrosilylation of SiH functional organosiloxanes with allyloxy-functional polyethers in the presence of a Pt catalyst. In this embodiment, component (A) is a silicone polyether having the structure represented by:

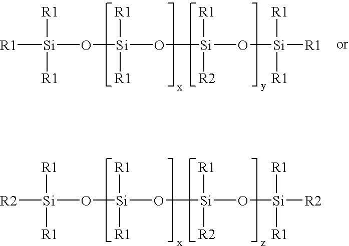

In these structures, RI represents an alkyl group containing 1-6 carbon atoms such as methyl, ethyl, propyl, butyl, pentyl, and hexyl; R2 represents the group—(CH₂)ₐO(C₂H₄O)_{b}(C₃H₆O)_{c}R3; x has a value of 1-1,000, alternatively 1-500, or alternatively 10-300; y has a value of 1-500, alternatively 1-100, or alternatively 2-50; z has a value of 1-500, or alternatively 1-100; a has a value of 3-6; b has a value of 4-20; c has a value of 0-5; and R3 is hydrogen, a methyl group, or an acyl group such as acetyl. Typically, R1 is methyl; b is 6-12; c is zero; and R3 is hydrogen.

Preferably, the rake type SPE the silicone polyether has a D/D′ ratio (i.e. x/y ratio) ranging from 5/1 to 50/1, alternatively from 10/1 to 30/1 or alternatively from 15/1 to 30/1.

In a second embodiment, component (A) is an (AB)ₙ block silicone polyether (polyorganosiloxane-polyoxyalkylene block copolymer) having the average formula;

—[R¹(RₛSiO)_{x′}(R₂SiR¹O)(CₘH₂ₘO)_{y′}]ₙ— [Formula 1]

where x′ and y′ are greater than 4, m is from 2 to 4 inclusive, n is greater than 2, R is independently a monovalent organic group containing 1 to 20 carbons, R¹ is a divalent hydrocarbon containing 2 to 30 carbons.

The siloxane block in Formula I is a predominately linear siloxane polymer having the formula (R₂SiO)_{x′}, wherein R is independently selected from a monovalent organic group, x′ is a integer greater than 4, alternatively x′ ranges from 20 to 100, or from 30 to 75.

The organic groups represented by R in the siloxane polymer are free of aliphatic unsaturation. These organic groups may be independently selected from monovalent hydrocarbon and monovalent halogenated hydrocarbon groups free of aliphatic unsaturation. These monovalent groups may have from 1 to 20 carbon atoms, alternatively 1 to 10 carbon atoms, and are exemplified by, but not limited to alkyl groups such as methyl, ethyl, propyl, butyl, pentyl, hexyl, heptyl, octyl, undecyl, and octadecyl; cycloalkyl such as cyclohexyl; aryl such as phenyl, tolyl, xylyl, benzyl, and 2-phenylethyl; and halogenated hydrocarbon groups such as 3,3,3-trifluoropropyl, 3-chloropropyl, and dichlorophenyl. At least 50 percent, alternatively at least 80%, of the organic groups in the organopolysiloxane may be methyl (denoted as Me). Typically, the siloxane block is a predominately linear polydimethylsiloxane having the formula (Me₂SiO)_{x′}, where x′ is as defined above.

The polyoxyalkylene block of the silicone polyether is represented by the formula (CₘH₂ₘO)_{y′} wherein m is from 2 to 4 inclusive, and y′ is greater than 4, alternatively y′ can range from 5 to 30, or alternatively from 5 to 25. The polyoxyalkylene block typically can comprise oxyethylene units (C₂H₄O)_{y′} oxypropylene units (C₃H₆O)_{y′}, oxybutylene units (C₄H₈O)_{y′}, of mixtures thereof. Typically, the polyoxyalkylene block comprises oxyethylene units (C₂4O)_{y′}.

At least one end of each polyoxyalkylene block in Formula I is linked to a siloxane block by a divalent organic group, designated R¹. This linkage is determined by the reaction employed to prepare the (AB)ₙ block silicone polyether copolymer. The divalent organic groups of R¹ may be independently selected from divalent hydrocarbons containing 2 to 30 carbons and divalent organofunctional hydrocarbons containing 2 to 30 carbons. Representative, non-limiting examples of such divalent hydrocarbon groups include; ethylene, propylene, butylene, pentylene, hexylene, heptylene, octylene, and the like. Representative, non-limiting examples of such divalent organofunctional hydrocarbons groups include acrylate and methacrylate. Typically, R¹ is propylene, (—CH₂CH₂CH₂—).

The (AB)ₙ block silicone polyethers are endblocked. The endblocking unit is also determined by the reaction employed to prepare the (AB)ₙ block silicone polyether copolymer, which is generally the residual reactive groups of the reactants used. For example, the (AB)ₙ block silicone polyether copolymers can be prepared by the metal catalyzed hydrosilylation reaction of a diallyl polyether (i.e. an allyl group is present on each molecular terminal end) with a SiH terminated polyorganosiloxane The resulting (AB)ₙ block silicone polyether copolymer would have polyoxyalkylene blocks linked to the silicone blocks via a propyleneoxy group (—CH₂CH₂CH₂O—), and using a slight molar excess of the allyl polyether would result in an allyl endblock unit (—CH₂CHCH₂). Alternative endblock units can result from the addition of other molecules in the reaction employed to prepare the (AB)ₙ block silicone polyether copolymer that are capable of reacting with the siloxane or polyether block intermediates. For example, the addition of organic compounds having mono-terminated aliphatic unsaturation (such as a mono allyl terminated polyether) will result in the endcapping of the (AB)ₙ block silicone polyether copolymer with that organic compound. Preferably, the endblocking units of the (AB)ₙ block silicone polyether is an allyl ether (CH₂═CHCH₂O—) or allyl polyether.

The molecular weights of the (AB)ₙ block silicone polyether copolymers will be determined by the number of repeating siloxane and polyoxyethylene blocks, as indicated by the subscript n in Formula I. Typically, the value of n is such to provide weight average molecular weights (M_{W}) to range from 1,500 to 150,000, alternatively, from 10,000 to 100,000.

The (AB)ₙ SPEs of the present vesicle compositions have a molar ratio of the total siloxane units to the polyoxyethylene units in the (AB)ₙ block silicone polyether. This molecular parameter is expressed by the value of x′/(x′+y′) in Formula I. The value of x′/(x′+y′) can vary from 0.4 to 0.9, or alternatively from 0.55 to 0.9.

The (AB)ₙ SPEs useful to prepare the vesicle compositions of the present invention can be prepared by any method known in the art for preparing such block copolymers. Typically however, the (AB)ₙ SPEs useful in the preparation of the vesicle compositions of the present invention are obtained from a method comprising reacting an SiH terminated organopolysiloxane with a polyoxyethylene having an unsaturated hydrocarbon group at each molecular terminal, in a hydrosilylation reaction, wherein the mole ratio of the unsaturated hydrocarbon groups to SiH in the reaction is at least 1:1.

### B) Water-Miscible Volatile Solvent Component

Component B) is a water-miscible volatile solvent. As used herein “water-miscible” means the solvent forms a dispersion with water at room temperature for at least several hours. “Volatile” means the solvent has a higher vapor pressure than water at various temperatures. As such, when the aqueous dispersion of the organopolysiloxane and solvent are subjected to conditions to remove the solvent, such as heating the dispersion under reduced pressures, the solvent is primarily removed first, allowing all or most of the water to remain in the composition.

Suitable water-miscible volatile solvents for vesicle dispersion preparation include organic solvents such as alcohols, ethers, glycols, esters, acids, halogenated hydrocarbons, diols. The organic solvents should be miscible with water at the proportion and lower in order to effectively disperse silicones and maintain stable and uniform dispersion overtime. For the purpose of illustration, water-miscible alcohols include method, ethanol, propanol, isopropanol, butanol, and higher hydrocarbon alcohols; ethers include gylcol ethers, methyl-ethyl ether, methyl isobutyl ether (MIBK), etc; glycols include propylene glycols, esters include esters of triglycerol, the esterification products of acid and alcohol; halogenated hydrocarbons include chloroform. Typically water-miscible organic solvents are solvents with relatively low boiling points (<100° C.) or high evaporation rate, so they may be removed under vacuum with ease. The most preferred water-miscible organic solvents for this invention are volatile alcohols including methanol, ethanol, isopropanol, and propanol. These alcohols can be removed from aqueous mixtures containing silicone vesicle dispersions via vacuum stripping at ambient temperature.

### C) Optional Silicone or Organic Oil Component

Optional component C) is a silicone or organic oil. The silicone can be any organopolysiloxane having the general formula RiSiO_{(4-1)/2} in which i has an average value of one to three and R is a monovalent organic group. The organopolysiloxane can be cyclic, linear, branched, and mixtures thereof.

In one embodiment, component C) is a volatile methyl siloxane (VMS) which includes low molecular weight linear and cyclic volatile methyl siloxanes. Volatile methyl siloxanes conforming to the CTFA definition of cyclomethicones are considered to be within the definition of low molecular weight siloxane.

Linear VMS have the formula (CH₃)₃SiO{(CH₃)₂SiO}_{f}Si(CH₃)₃. The value off is 0-10. Cyclic VMS have the formula {(CH3)2SiO}_{g}. The value of g is 3-6. Preferably, these volatile methyl siloxanes have a molecular weight of less than 1,000; a boiling point less than 250° C.; and a viscosity of 0.65 to 5.0 centistoke (mm²/s), generally not greater than 5.0 centistoke (mm²/s).

Representative linear volatile methyl siloxanes are hexamethyldisiloxane (MM) with a boiling point of 100° C., viscosity of 0.65 mm²/s, and formula Me₃SiOSiMe₃; octamethyltrisiloxane (MDM) with a boiling point of 152° C., viscosity of 1.04 mm²/s, and formula Me₃SiOMe₂SiOSiMe₃; decamethyltetrasiloxane (MD₂M) with a boiling point of 194° C., viscosity of 1.53 mm²/s, and formula Me₃SiO(Me₂SiO)₂SiMe₃; dodecamethylpentasiloxane (MD₃M) with a boiling point of 229° C., viscosity of 2.06 mm²/s, and formula Me₃SiO(Me₂SiO)₃SiMe₃; tetradecamethylhexasiloxane (MD₄M) with a boiling point of 245° C., viscosity of 2.63 mm²/s, and formula Me₃SiO(Me₂SiO)₄SiMe₃; and hexadecamethylheptasiloxane (M₅DM) with a boiling point of 270° C., viscosity of 3.24 mm²/s, and formula Me₃SiO(Me₂SiO)₅SiMe₃.

Representative cyclic volatile methyl siloxanes are hexamethylcyclotrisiloxane (D3), a solid with a boiling point of 134° C., a molecular weight of 223, and formula {(Me₂)SiO}₃; octamethylcyclotetrasiloxane (D₄) with a boiling point of 176° C., viscosity of 2.3 mm²/s, a molecular weight of 297, and formula {(Me₂)SiO}₄; decamethylcyclopentasiloxane (D₅) with a boiling point of 210° C., viscosity of 3.87 mm²/s, a molecular weight of 371, and formula {(Me₂)SiO}₅; and dodecamethylcyclohexasiloxane (D₆) with a boiling point of 245° C., viscosity of 6.62 mm²/s, a molecular weight of 445, and formula {(Me₂)SiO}₆.

The silicone selected as component C) can be any polydiorganosiloxane fluid, gum, or mixtures thereof. If the polyorganosiloxane has a molecular weight equal to or greater than 1000, it can be blended with the volatile methyl siloxanes described above. The polydiorganosiloxane gums suitable for the present invention are essentially composed of dimethylsiloxane units with the other units being represented by monomethylsiloxane, trimethylsiloxane, methylvinylsiloxane, methylethylsiloxane, diethylsiloxane, methylphenylsiloxane, diphenylsiloxane, ethylphenylsiloxane, vinylethylsiloxane, phenylvinylsiloxane, 3,3,3-trifluoropropylmethylsiloxane, dimethylphenylsiloxane, methylphenylvinylsiloxane, dimethylethylsiloxane, 3,3,3-trifluoropropyldimethylsiloxane, mono-3,3,3-trifluoropropylsiloxane, aminoalkylsiloxane, monophenylsiloxane, monovinylsiloxane and the like.

When component C) is an organic oil, it may be selected from any organic oil known in the art suitable for use in the preparation of personal, household, or healthcare formulations. Suitable organic oils include, but are not limited to, natural oils such as coconut oil; hydrocarbons such as mineral oil and hydrogenated polyisobutene; fatty alcohols such as octyldodecanol; esters such as C12-C15 alkyl benzoate; diesters such as propylene dipelarganate; and triesters, such as glyceryl trioctanoate. The organic oil components can also be mixture of low viscosity and high viscosity oils. Suitable low viscosity oils have a viscosity of 5 to 100 mPa·s at 25° C., and are generally esters having the structure RCO-OR¹ wherein RCO represents the carboxylic acid radical and wherein OR′ is an alcohol residue. Examples of these low viscosity oils include isotridecyl isononanoate, PEG-4 diheptanoate, isostearyl neopentanoate, tridecyl neopentanoate, cetyl octanoate, cetyl palmitate, cetyl ricinoleate, cetyl stearate, cetyl myristate, coco-dicaprylate/caprate, decyl isostearate, isodecyl oleate, isodecyl neopentanoate, isohexyl neopentanoate, octyl palmitate, dioctyl malate, tridecyl octanoate, myristyl myristate, octododecanol, or mixtures of octyldodecanol, acetylated lanolin alcohol, cetyl acetate, isododecanol, polyglyceryl-3-diisostearate, or mixtures thereof. The high viscosity surface oils generally have a viscosity of 200-1,000,000 mPa·s at 25° C., preferably a viscosity of 100,000-250,000 mPa·s. Surface oils include castor oil, lanolin and lanolin derivatives, triisocetyl citrate, sorbitan sesquioleate, C10-18 triglycerides, caprylic/capric/triglycerides, coconut oil, corn oil, cottonseed oil, glyceryl triacetyl hydroxystearate, glyceryl triacetyl ricinoleate, glyceryl trioctanoate, hydrogenated castor oil, linseed oil, mink oil, olive oil, palm oil, illipe butter, rapeseed oil, soybean oil, sunflower seed oil, tallow, tricaprin, trihydroxystearin, triisostearin, trilaurin, trilinolein, trimyristin, triolein, tripalmitin, tristearin, walnut oil, wheat germ oil, cholesterol, or mixtures thereof. Mention may be made, among the optional other non-silicone fatty substances, of mineral oils, such as liquid paraffin or liquid petroleum, of animal oils, such as perhydrosqualene or arara oil, or alternatively of vegetable oils, such as sweet almond, calophyllum, palm, castor, avocado, jojaba, olive or cereal germ oil. It is also possible to use esters of lanolic acid, of oleic acid, of lauric acid, of stearic acid or of myristic acid, for example; alcohols, such as oleyl alcohol, linoleyl or linolenyl alcohol, isostearyl alcohol or octyldodecanol; or acetylglycerides, octanoates, decanoates or ricinoleates of alcohols or of polyalcohols. It is alternatively possible to use hydrogenated oils which are solid at 25° C., such as hydrogenated castor, palm or coconut oils, or hydrogenated tallow; mono-, di-, tri- or sucroglycerides; lanolins; or fatty esters which are solid at 25° C.

The amount of components A), B), and C) can vary in the process, but typically range as follows;
A) 2 to 50 wt%, alternatively 2 to 25 wt %, or alternatively 1 to 20 wt%,
B) 1 to 50 wt%, alternatively 2 to 25 wt %, or alternatively 2 to 15 wt%,
C) 0 to 50 wt %, alternatively 1 to 20 wt %, or alternatively 2 to 10 wt%,
   and sufficient amount of water to provide the sum of the wt % of A), B), and C) and water content to equal 100%.

The order of combining components A), B), and C) with water is not critical, but typically A), B), and C) are first combined and then added with water to form an aqueous dispersions of components A)-C).

Step II in the process of the present invention is mixing the aqueous dispersion formed in Step I to form vesicles. There are no special requirements or conditions needed to effect the mixing and formation of vesicles. Mixing techniques can be simple stirring, homogenizing, sonalating, and other mixing techniques known in the art to effect the formation of vesicles in aqueous dispersions. The mixing can be conducted in a batch, semi-continuous, or continuous process.

The formation of vesicles can be confirmed by techniques common in the state of the art. Typically, vesicles have a lamellar phase structure which exhibit birefringence when examined with a cross polarizing microscope. Alternatively, the formation of vesicles can be demonstrated by Cyro-Transmission Electron Microscopy (Cryo-TEM) techniques. Particle size measurements can also be used to indicate that the organopolysiloxanes are sufficiently dispersed in aqueous medium typical of vesicle sizes. For example, average particle sizes of less than 0.500 μm (micrometers), are typical for dispersed vesicles. Vesicles having an average particle size of less than 0.200 μm, or 0.100 μm are possible with the teachings of the present invention.

Step III in the process of the present invention is optional, and involves removing the water miscible volatile solvent, component B). Typically, the water miscible volatile solvent is removed by known techniques in the art, such as subjecting the vesicle composition to reduced pressures, while optionally heating the composition. Devices illustrative of such techniques include rotary evaporators and thin film strippers.

Step IV) in the process of the present invention involves admixing to the vesicle dispersion component D), a hydrophobic active. As used herein “hydrophobic active” encompasses any hydrophobic composition that may be used in a personal or healthcare composition to effect a desired cosmetic (personal care) or pharmaceutical (healthcare) benefit. Component D) may be a single hydrophobic active, or it may also be a mixture of several materials, providing the overall mixture is considered hydrophobic and contains at least one “active” component. Typically, component D) is selected from;
D′) a silicone oil,
D″) a personal care active,
D′″) a healthcare active,
   and mixtures thereof.
   When component D) contains D′) a silicone oil, the silicone oil may be selected from any of the silicone oils described above as component C). Preferred silicone oils include polydimethylsiloxanes, such as Dow Corning® 200 fluids (INCI name dimethicone), dimethylcyclosiloxanes, such as Dow Corning® 245 Fluid (INCI name cyclopentasiloxane) and phenyl functional siloxanes, such as Dow Corning® 556 Fluid (INCI name phenyltrimethicone).

### D″) Personal or D′″) Healthcare Active Component

Component D) is a personal care or healthcare active. As used herein, a “personal care active” means any compound or mixtures of compounds that are known in the art as additives in the personal care formulations that are typically added for the purpose of treating hair or skin to provide a cosmetic and/or aesthetic benefit. A “healthcare active” means any compound or mixtures of compounds that are known in the art to provide a pharmaceutical or medical benefit. Thus, “healthcare active” include materials consider as an active ingredient or active drug ingredient as generally used and defined by the United States Department of Health & Human Services Food and Drug Administration, contained in Title 21, Chapter I, of the Code of Federal Regulations, Parts 200-299 and Parts 300-499.

Thus, active ingredient can include any component that is intended to furnish pharmacological activity or other direct effect in the diagnosis, cure, nitigation, treatment, or prevention of disease, or to affect the structure or any function of the body of a human or other animals. The phrase can include those components that may undergo chemical change in the manufacture of drug products and be present in drug products in a modified form intended to furnish the specified activity or effect.

Some representative examples of active ingredients include; drugs, vitamins, minerals; hormones; topical antimicrobial agents such as antibiotic active ingredients, antifungal active ingredients for the treatment of athlete's foot, jock itch, or ringworm, and acne active ingredients; astringent active ingredients; deodorant active ingredients; wart remover active ingredients; corn and callus remover active ingredients; pediculicide active ingredients for the treatment of head, pubic (crab), and body lice; active ingredients for the control of dandruff, seborrheic dermatitis, or psoriasis; and sunburn prevention and treatment agents.

By forming into silicone vesicles, these active ingredients are efficiently kept on the skin and result in a longer-lasting effect of the product. Further, we can control the stimulation or inhibition of transdermal absorption of active ingredients by formulating some additives. For example, some active ingredients are efficiently absorbed through the skin by formulating ethanol as volatile content, esters or menthol as stimulator of transdermal absorption. Especially, combination of aqueous active ingredients with silicone vesicles containing oil-soluble ones have advantages to stimulate transdermal absorption of these ingredients.

Useful active ingredients for use in processes according to the invention include vitamins and its derivatives, including “pro-vitamins”. Vitamins useful herein include, but are not limited to, Vitamin A₁, retinol, C₂-C₁₈ esters of retinol, vitamin E, tocopherol, esters of vitamin E, and mixtures thereof. Retinol includes trans-retinol, 1,3-cis-retinol, 11-cis-retinol, 9-cis-retinol, and 3,4-didehydro-retinol, Vitamin C and its derivatives, Vitamin B₁, Vitamin B₂, Pro Vitamin B5, panthenol, Vitamin B₆, Vitamin B₁₂, niacin, folic acid, biotin, and pantothenic acid. Other suitable vitamins and the INCI names for the vitamins considered included herein are ascorbyl dipalmitate, ascorbyl methylsilanol pectinate, ascorbyl palmitate, ascorbyl stearate, ascorbyl glucocide, sodium ascorbyl phosphate, sodium ascorbate, disodium ascorbyl sulfate, potassium (ascorbyl/tocopheryl) phosphate.

RETINOL, it should be noted, is an International Nomenclature Cosmetic Ingredient Name (INCI) designated by The Cosmetic, Toiletry, and Fragrance Association (CTFA), Washington DC, for vitamin A. Other suitable vitamins and the INCI names for the vitamins considered included herein are RETINYL ACETATE, RETINYL PALMITATE, RETINYL PROPIONATE, α-TOCOPHEROL, TOCOPHERSOLAN, TOCOPHERYL ACETATE, TOCOPHERYL LINOLEATE, TOCOPHERYL NICOTINATE, and TOCOPHERYL SUCCINATE.

Some examples of commercially available products suitable for use herein are Vitamin A Acetate and Vitamin C esters, both products of Fluka Chemie AG, Buchs, Switzerland; COVI-OX T-50, a vitamin E product of Henkel Corporation, La Grange, Ill.; COVI-OX T-70, another vitamin E product of Henkel Corporation, La Grange, Ill.; and vitamin E Acetate, a product of Roche Vitamins & Fine Chemicals, Nutley, N.J.

The active ingredient used in processes according to the invention can be an active drug ingredient. Representative examples of some suitable active drug ingredients which can be used are hydrocortisone, ketoprofen, timolol, pilocarpine, adriamycin, mitomycin C, morphine, hydromorphone, diltiazem, theophylline, doxorubicin, daunorubicin, heparin, penicillin G, carbenicillin, cephalothin, cefoxitin, cefotaxime, 5-fluorouracil, cytarabine, 6-azauridine, 6-thioguanine, vinblastine, vincristine, bleomycin sulfate, aurothioglucose, suramin, mebendazole, clonidine, scopolamine, propranolol, phenylpropanolamine hydrochloride, ouabain, atropine, haloperidol, isosorbide, nitroglycerin, ibuprofen, ubiquinones, indomethacin, prostaglandins, naproxen, salbutamol, guanabenz, labetalol, pheniramine,.metrifonate, and steroids.

Considered to be included herein as active drug ingredients for purposes of the present invention are antiacne agents such as benzoyl peroxide and tretinoin; antibacterial agents such as chlorohexadiene gluconate; antifungal agents such as miconazole nitrate; anti-inflammatory agents; corticosteroidal drugs; non-steroidal anti-inflammatory agents such as diclofenac; antipsoriasis agents such as clobetasol propionate; anesthetic agents such as lidocaine; antipruritic agents; antidermatitis agents; and agents generally considered barrier films.

The active component D) of the present invention can be a protein, such as an enzyme. The internal inclusion of enzymes in the silicone vesicle have advantages to prevent enzymes from deactivating and maintain bioactive effects of enzymes for longer time. Enzymes include, but are not limited to, commercially available types, improved types, recombinant types, wild types, variants not found in nature, and mixtures thereof. For example, suitable enzymes include hydrolases, cutinases, oxidases, transferases, reductases, hemicellulases, esterases, isomerases, pectinases, lactases, peroxidases, laccases, catalases, and mixtures thereof. Hydrolases include, but are not limited to, proteases (bacterial, fungal, acid, neutral or alkaline), amylases (alpha or beta), lipases, mannanases, cellulases, collagenases, lisozymes, superoxide dismutase, catalase, and mixtures thereof. Said protease include, but are not limited to, trypsin, chymotrypsin, pepsin, pancreatin and other mammalian enzymes; papain, bromelain and other botanical enzymes; subtilisin, epidermin, nisin, naringinase(L-rhammnosidase) urokinase and other bacterial enzymes. Said lipase include, but are not limited to, triacyl-glycerol lipases, monoacyl-glycerol lipases, lipoprotein lipases, e.g. steapsin, erepsin, pepsin, other mammalian, botanical, bacterial lipases and purified ones. Natural papain is preferred as said enzyme. Further, stimulating hormones, e.g. insulin, can be used together with these enzymes to boost the effectiveness of them.

Component D) may also be a sunscreen agent. The sunscreen agent can be selected from any sunscreen agent known in the art to protect skin from the harmful effects of exposure to sunlight. The sunscreen compound is typically chosen from an organic compound, an inorganic compound, or mixtures thereof that absorbs ultraviolet (UV) light. Thus, representative non limiting examples that can be used as the sunscreen agent include; Aminobenzoic Acid, Cinoxate, Diethanolarnine Methoxycinnamate, Digalloyl Trioleate, Dioxybenzone, Ethyl 4-[bis(Hydroxypropyl)] Aminobenzoate, Glyceryl Aminobenzoate, Homosalate, Lawsone with Dihydroxyacetone, Menthyl Anthranilate, Octocrylene, Octyl Methoxycinnamate, Octyl Salicylate, Oxybenzone, Padimate O, Phenylbenzimidazole Sulfonic Acid, Red Petrolatum, Sulisobenzone, Titanium Dioxide, and Trolamine Salicylate, cetaminosalol, Allatoin PABA, Benzalphthalide, Benzophenone, Benzophenone 1-12, 3-Benzylidene Camphor, Benzylidenecamphor Hydrolyzed Collagen Sulfonamide, Benzylidene Camphor Sulfonic Acid, Benzyl Salicylate, Bornelone, Bumetriozole, Butyl Methoxydibenzoylmethane, Butyl PABA, Ceria/Silica, Ceria/Silica Talc, Cinoxate, DEA-Methoxycinnamate, Dibenzoxazol Naphthalene, Di-t-Butyl Hydroxybenzylidene Camphor, Digalloyl Trioleate, Diisopropyl Methyl Cinnamate, Dimethyl PABA Ethyl Cetearyldimonium Tosylate, Dioctyl Butamido Triazone, Diphenyl Carbomethoxy Acetoxy Naphthopyran, Disodium Bisethylphenyl Tiamminotriazine Stilbenedisulfonate, Disodium Distyrylbiphenyl Triaminotriazine Stilbenedisulfonate, Disodium Distyrylbiphenyl Disulfonate, Drometrizole, Drometrizole Trisiloxane, Ethyl Dihydroxypropyl PABA, Ethyl Diisopropylcinnamate, Ethyl Methoxycinnamate, Ethyl PABA, Ethyl Urocanate, Etrocrylene Ferulic Acid, Glyceryl Octanoate Dimethoxycinnamate, Glyceryl PABA, Glycol Salicylate, Homosalate, Isoamyl p-Methoxycinnamate, Isopropylbenzyl Salicylate, Isopropyl Dibenzolylmethane, Isopropyl Methoxycinnamate, Menthyl Anthranilate, Menthyl Salicylate, 4-Methylbenzylidene, Camphor, Octocrylene, Octrizole, Octyl Dimethyl PABA, Octyl Methoxycinnamate, Octyl Salicylate, Octyl Triazone, PABA, PEG-25 PABA, Pentyl Dimethyl PABA, Phenylbenzimidazole Sulfonic Acid, Polyacrylamidomethyl Benzylidene Camphor, Potassium Methoxycinnamate, Potassium Phenylbenzimidazole Sulfonate, Red Petrolatum, Sodium Phenylbenzimidazole Sulfonate, Sodium Urocanate, TEA-Phenylbenzimidazole Sulfonate, TEA-Salicylate, Terephthalylidene Dicamphor Sulfonic Acid, Titanium Dioxide, Zinc Dioxide, Serium Dioxide, TriPABA Panthenol, Urocanic Acid, and VA/Crotonates/Methacryloxybenzophenone-1 Copolymer.

These sunscreen agent can be selected one or combination of more than one. Further, the silicone vesicle can contain one sunscreen agent in inner phase, and another in outer phase, e g containing oil-soluble sunscreen agent in inner phase and water-dispersible one in outer phase of this silicone vesicle. In this usage, the silicone vesicle is useful to stabilize the combination of different sunscreens for some organic sunscreen agents are colored by contacting with Titanium dioxide directly.

Alternatively, the sunscreen agent is a cinnamate based organic compound, or alternatively, the sunscreen agent is octyl methoxycinnamate, such as Parsol MCX or Uvinul® MC 80 an ester of para-methoxycinnamic acid and 2-ethylhexanol.

Component D) may also be a fragrance or perfume. The perfume can be any perfume or fragrance active ingredient commonly used in the perfume industry. These compositions typically belong to a variety of chemical classes, as varied as alcohols, aldehydes, ketones, esters, ethers, acetates, nitrites, terpenic hydrocarbons, heterocyclic nitrogen or sulfur containing compounds, as well as essential oils of natural or synthetic origin. Many of these perfume ingredients are described in detail in standard textbook references such as Perfume and Flavour Chemicals, 1969, S. Arctander, Montclair, N.J.

Fragrances may be exemplified by, but not limited to, perfume ketones and perfume aldehydes. Illustrative of the perfume ketones are buccoxime; iso jasmone; methyl beta naphthyl ketone; musk indanone; tonalid/musk plus; Alpha-Damascone, Beta-Damascone, Delta-Damascone, Iso-Damascone, Damascenone, Damarose, Methyl-Dihydrojasmonate, Menthone, Carvone, Camphor, Fenchone, Alpha-lonone, Beta-lonone, Gamma-Methyl so-called Ionone, Fleuramone, Dihydrojasmone, Cis-Jasmone, Iso-E-Super, Methyl-Cedrenyl-ketone or Methyl- Cedrylone, Acetophenone, Methyl-Acetophenone, Para-Methoxy-Acetophenone, Methyl-Beta-Naphtyl-Ketone, Benzyl-Acetone, Benzophenone, Para-Hydroxy-Phenyl-Butanone, Celery Ketone or Livescone, 6-Isopropyldecahydro-2-naphtone, Dimethyl-Octenone, Freskomenthe, 4-(1-Ethoxyvinyl)-3,3,5,5,-tetramethyl-Cyclohexanone, Methyl-Heptenone, 2-(2-(4-Methyl-3-cyclohexen-1-yl)propyl)-cyclopentan one, 1-(p-Menthen-6(2)-yl)-1-propanone, 4-(4-Hydroxy-3-methoxyphenyl)-2-butanone, 2-Acetyl-3,3-Dimethyl-Norbomane, 6,7-Dihydro-1,1,2,3,3-Pentamethyl-4(5H)-Indanone, 4-Damascol, Dulcinyl or Cassione, Gelsone, Hexalon, Isocyclemone E, Methyl Cyclocitrone, Methyl-Lavender-Ketone, Orivon, Para-tertiary-Butyl-Cyclohexanone, Verdone, Delphone, Muscone, Neobutenone, Plicatone, Veloutone, 2,4,4,7-Tetramethyl-oct-6-en-3-one, and Tetrameran.

More preferably, the perfume ketones are selected for its odor character from Alpha Damascone, Delta Damascone, Iso Damascone, Carvone, Gamma-Methyl-lonone, Iso-E-Super, 2,4,4,7-Tetramethyl-oct-6-en-3-one, Benzyl Acetone, Beta Damascone, Damascenone, methyl dihydrojasmonate, methyl cedrylone, and mixtures thereof.

Preferably, the perfume aldehyde is selected for its odor character from adoxal; anisic aldehyde; cymal; ethyl vanillin; florhydral; helional; heliotropin; hydroxycitronellal; koavone; lauric aldehyde; lyral; methyl nonyl acetaldehyde; P. T. bucinal; phenyl acetaldehyde; undecylenic aldehyde; vanillin; 2,6,10-trimethyl-9-undecenal, 3-dodecen-1-al, alpha-n-amyl cinnamic aldehyde, 4-methoxybenzaldehyde, benzaldehyde, 3-(4-tert butylphenyl)-propanal, 2-methyl-3-(para-methoxyphenyl propanal, 2-methyl-4-(2,6,6-trimethyl-2(1)-cyclohexen- 1-yl) butanal, 3-phenyl-2-propenal, cis-/trans-3,7-dimethyl-2,6-octadien-1-al, 3,7-dimethyl-6-octen-1-al, [(3,7-dimethyl-6-octenyl)oxy] acetaldehyde, 4-isopropylbenzyaldehyde, 1,2,3,4,5,6,7,8-octahydro-8,8-dimethy]-2-naphthaldehyde , 2,4-dimethyl-3-cyclohexen-1-carboxaldehyde, 2-methyl-3-(isopropylphenyl)propanal, 1-decanal; decyl aldehyde, 2,6-dimethyl-5-heptenal, 4-(tricyclo[5.2.1.0(2,6))-decylidene-8)-butanal, octahydro-4,7-methano-1H- indenecarboxaldehyde, 3-ethoxy-4-hydroxy benzaldehyde, para-ethyl-alpha, alpha-di methyl hydrocinnamaldehyde, alpha-methyl-3,4-(methylenedioxy)-hydrocinnamaldehyde, 3,4-methylenedioxybenzaldehyde, alpha-n-hexyl cinnamic aldehyde, m-cymene-7-carboxaldehyde, alpha-methyl phenyl acetaldehyde, 7-hydroxy-3,7-dimethyl octanal, Undecenal, 2,4,6-trimethyl-3-cyclohexene-1-carboxaldehyde, 4-(3)(4-methyl-3-pentenyl)-3-cyclohexen-carboxaldehyde, 1-dodecanal, 2,4-dimethyl cyclohexene-3-carboxaldehyde, 4-(4-hydroxy4-methyl pentyl)-3-cylohexene-1-carboxaldehyde, 7-methoxy-3,7-dimethyloctan-1-al, 2-methyl undecanal, 2-methyl decanal, 1-nonanal, 1-octanal, 2,6,10-trimethyl-5,9-undecadienal, 2-methyl-3-(4-tertbutyl)propanal, dihydrocinnamic aldehyde, 1-methyl-4-(4-methyl-3-pentenyl)-3-cyclohexene-1-carboxaldehyde, 5 or 6 methoxyl 0 Hexahydro-4,7-methanoindan-1 or 2- carboxaldehyde, 3,7-dimethyloctan-1-al, 1-undecanal, 10-undecen-1-al, 4-hydroxy-3-methoxy benzaldehyde, 1-methyl-3-(4-methylpentyl)-3-cyclhexenecarboxaldehyde, 7-hydroxy-3,7-dimethyl-octanal, trans-4-decenal, 2,6-nonadienal, paratolylacetaldehyde; 4-methylphenylacetaldehyde, 2-methyl-4-(2,6,6-trimethyl-1-cyclohexen-1-yl)-2-butenal, ortho-methoxycinnamic aldehyde, 3,5,6-trimethyl-3-cyclohexene carboxaldehyde, 3,7-dimethyl-2-methylene-6-octenal, phenoxyacetaldehyde, 5,9-dimethyl-4,8-decadienal, peony aldehyde (6,10-dimethyl-3-oxa-5,9-undecadien-1-al), hexahydro-4,7-methanoindan-1-carboxaldehyde, 2-methyl octanal, alpha-methyl-4-(1-methyl ethyl) benzene acetaldehyde, 6,6-dimethyl-2-norpinene-2-propionaldehyde, para methyl phenoxy acetaldehyde, 2-methyl-3-phenyl-2-propen-1-al, 3,5,5-trimethyl hexanal, Hexahydro-8,8-dimethyl-2-naphthaldehyde, 3-propyl-bicyclo[2.2.1]-hept-5-ene-2-carbaldehyde, 9-decenal, 3-methyl-5-phenyl-1-pentanal, methylnonyl acetaldehyde, hexanal, trans-2-hexenal, 1-p-menthene-q-carboxaldehyde and mixtures thereof.

More preferred aldehydes are selected for their odor character from 1-decanal, benzaldehyde, florhydral, 2,4-dimethyl-3-cyclohexen-1-carboxaldehyde; cis/trans-3,7-dimethyl-2,6-octadien-1-al; heliotropin; 2,4,6-trimethyl-3-cyclohexene-1-carboxal dehyde; 2,6-nonadienal; alpha-n-amyl cinnamic aldehyde, alpha-n-hexyl cinnamic aldehyde, P.T. Bucinal, lyral, cymal, methyl nonyl acetaldehyde, hexanal, trans-2-hexenal, and mixture thereof.

In the above list of perfume ingredients, some are commercial names conventionally known to one skilled in the art, and also includes isomers. Such isomers are also suitable for use in the present invention.

Component D) may also be one or more plant extract. Examples of these components are as follows: Ashitaba extract, avocado extract, hydrangea extract, Althea extract, Arnica extract, aloe extract, apricot extract, apricot kernel extract, Ginkgo Biloba extract, fennel extract, turmeric[Curcuma] extract, oolong tea extract, rose fruit extract, Echinacea extract, Scutellaria root extract, Phellodendro bark extract, Japanese Coptis extract, Barley extract, Hyperium extract, White Nettle extract, Watercress extract, Orange extract, Dehydrated saltwater, seaweed extract, hydrolyzed elastin, hydrolyzed wheat powder, hydrolyzed silk, Chamomile extract, Carrot extract, Artemisia extract, Glycyrrhiza extract, hibiscustea extract, Pyracantha Fortuneana Fruit extract, Kiwi extract, Cinchona extract, cucumber extract, guanocine, Gardenia extract, Sasa Albo-marginata extract, Sophora root extract, Walnut extract, Grapefruit extract, Clematis extract, Chlorella extract, mulberry extract, Gentiana extract, black tea extract, yeast extract, burdock extract, rice bran ferment extract, rice germ oil, comfrey extract, collagen, cowberry extract, Gardenia extract, Asiasarum Root extract, Family of Bupleurum extract, umbilical cord extract, Salvia extract, Saponaria extract, Bamboo extract, Crataegus fruit extract, Zanthoxylum fruit extract, shiitake extract, Rehmannia root extract, gromwell extract, Perilla extract, linden extract, Filipendula extract, peony extract, Calamus Root extract, white birch extract, Horsetail extract, Hedera Helix(Ivy) extract, hawthorn extract, Sambucus nigra extract, Achillea millefolium extract, Mentha piperita extract, sage extract, mallow extract, Cnidium officinale Root extract, Japanese green gentian extract, soybean extract, jujube extract, thyme extract, tea extract, clove extract, Gramineae imperata cyrillo extract, Citrus unshiu peel extract Japanese Angellica Root extract, Calendula extract, Peach Kernel extract, Bitter orange peel extract, Houttuyna cordata extract, tomato extract, natto extract, Ginseng extract, Green tea extract (camellica sinesis), grape seed extract, garlic extract, wild rose extract, hibiscus extract, Ophiopogon tuber extarct, Nelumbo nucifera extract, parsley extract, honey, hamamelis extract, Parietaria extract, Isodonis herba extract, bisabolol extract, Loquat extract, coltsfoot extract, butterbur extract, Porid cocos wolf extract, extract of butcher's broom, grape extract, propolis extract, luffa extract, safflower extract, peppermintextract, linden tree extract, Paeonia extract, hop extract, pine tree extract, horse chestnut extract, Mizu-bashou [Lysichiton camtschatcese]extract, Mukurossi peel extract, Melissa extract, peach extract, cornflower extract, eucalyptus extract, saxifrage extract, citron extract, coix extract, mugwort extract, lavender extract, apple extract, lettuce extract, lemon extract, Chinese milk vetch extract, rose extract, rosemary extract, Roman Chamomile extract, and royal jelly extract.

The amount of component D) can vary in the process, but typically range as follows; 0.05 to 40 wt%, alternatively 0.1 to 30 wt %, or alternatively 0.1 to 20 wt%, of the vesicle composition. That is, the wt% of A), B), C), D), and water content to equal 100% and the ranges for A), B), and C) are as defined above.

The “admixing” in step IV) involves adding and mixing component D) to the vesicle dispersion formed in step III) of the present process. The addition and mixing of component D) to the vesicle dispersion formed in step III) may occur in one step (that is simultaneous addition and mixing), or alternatively, may occur in two steps. When two steps are used for admixing of step IV), component D) is first added to the vesicle dispersion using simple mixing or stirring techniques, and then the resulting mixture is subjected to a shear mixing process. Representative, non-limiting examples of such shear mixing processes include homogenizers, sonalators, Microfluidizers, Roto-Stators, and other techniques known in the art effect shear mixing.

Although not wishing to be bound by any theory, the present inventors believe admixing of component D) to the pre-formed vesicle dispersion, allows for the hydrophobic active to become entrapped within the hydrophobic silicone bilayer of the vesicle structures. The bilayers of the silicone vesicles have sufficient robustness to withstand shear forces. The shear mixing may further reduce particle size of the vesicles structures, and leads to greater storage stability of the entrapped actives.

This post-load/shear method is thus useful for encapsulating non-silicone, personal care actives including vitamins, sunscreens, fragrances with silicone vesicles. Organic actives may be loaded directly, or preferably as a mixture of the organic active with a silicone fluid into silicone vesicles. The use of a silicone fluid for loading organic actives may result in loaded silicone vesicles with better long term stability in water or water containing personal care formulations. The vesicle containing actives may be further incorporated into personal care formulations such as; an antiperspirant, deodorant, skin cream, skin care lotion, moisturizer, facial treatment, wrinkle remover, facial cleansers, bath oils, sunscreens, pre-shave, after-shave lotions, liquid soap, shaving soap, shaving lather, hair shampoo, hair conditioner, hair spray, mousse, permanent, hair cuticle coat, make-up, color cosmetic, foundation, blush, lipstick, lip balm, eyeliner, mascara, nail polishes, and powders.

### EXAMPLES

These examples are intended to illustrate the invention to one of ordinary skill in the art and should not be interpreted as limiting the scope of the invention set forth in the claims.

### Example 1 (Reference)

### Preparation of Neat Silicone Vesicles from Rake SPE

A silicone vesicle in water dispersion was prepared from hydrophobic rake silicone polyether (referenced as rake SPE herein), which was a silicone polyether having a structure of MD₉₄D^{(EO12)}₆M. This rake SPE was the reaction product of MD₉₄D₆M and mono-allyl polyether, specifically a salt-free version of mono-allyl polyether to yield a SPE with high clarity. Rake SPE's prepared from commercially found mono-allyl polyethers (e.g. AE501 from Dow Chemical) were also used for the preparation of silicone vesicles in this invention. The silicone vesicle in water dispersion was prepared according to the methods described in WO2005/103157.

Alternatively, the method for preparing the neat vesicle was prepared by first adding SPE into alcohol with continuous mixing. Then, water was gradually added with continuous stirring. The resulting final mixture was a homogenous dispersion. This dispersion was then processed through a high shear device like Microfluidizer or equivalent to reduce the vesicle size. The processed dispersion was further stripped under vacuum at ambient temperature to remove volatile alcohol using a Rotovapor. The final vesicle is a translucent dispersion in water with an average particle size of 0.072 μm, as measured by Nanotrac particle analyzer.

TABLE 1
Neat silicone vesicles from rake SPE

| | Example # |
|---|---|
| | #1A |
| Description | Neat silicone vesicle in water |
| SPE type for vesicle | 20048-55 rake SPE |
| Wt. % SPE in vesicle dispersion | 20.0% |
| Appearance | Uniform, translucent dispersion |
| Mv Avg size, μm | 0.072 |
| D(v, 0.5), μm | 0.070 |
| D(v, 0.9), μm | 0.098 |

A separate batch of neat silicone vesicle in water dispersion was prepared from the same rake SPE. A slightly different alcohol/water composition was used. The final vesicle dispersion has an average particle size of about 0.150 μm in diameter.

TABLE 2
Neat silicone vesicles from rake SPE

| | Example # |
|---|---|
| | #1B |
| Description | Neat silicone vesicles in water |
| SPE type for vesicle | rake SPE |
| Wt % SPE in dispersion | 19.50% |
| Appearance | Uniform translucent dispersion |
| Mv avg. size, μm | 0.150 |
| D(v, 0.5), μm | 0.134 |
| D(v, 0.9), μm | 0.249 |

### Example 2

### Preparation of Silicone Fluid Emollients Containing Silicone Vesicles

Some silicone fluids are known to provide excellent emollient benefits in personal care formulations, however, these silicone fluids are hydrophobic oils and do not self-disperse in aqueous medium. This example demonstrates that silicone fluid emollients can be incorporated into silicone vesicles and become a stable homogeneous phase in water following the method described in this invention.

Illustrated in the followings are three silicone fluid emollient containing silicone vesicles in water. DC 200 fluid, 10 cSt is a polydimethylsiloxane based silicone fluid at 10 cSt viscosity. DC 556 fluid is phenyl(trimethylsiloxyl) siloxane and DC345 fluid is a dodecylmethylhexacyclosiloxane. The starting silicone vesicle is Reference Example #1A, which contains about 20.0 wt % SPE as vesicles and the balance is water continuous phase. The preparation of the silicone vesicle is shown in the previous section.

These examples were prepared according to the following procedure:
1. Incorporate silicone fluid into silicone vesicles in water dispersion,
2. Mix or shake to disperse silicone fluid using mechanical stirrer, shaker or vibrator,
3. Subject the above mixture to Microfluidizer® or equivalent high-shear device at a pre-determined pressure settings (10,000 psi in the case of Microfluidizer®),
4. Return the effluent of Microfluidizer® processed mixture to one more pass, to give a total of 2 passes through the Microfluidizer®
5. Inspect the appearance and the particle size distribution of the final mixture.

The composition and properties of silicone fluid containing silicone vesicle in water dispersion are shown in the following table. The wt % payload is the amount of actives (silicone fluids in this case) divided by the total of silicone vesicles and actives.

TABLE 3
Silicone fluids containing vesicles by post-load method

| | Example # | | |
|---|---|---|---|
| | 2A | 2B | 2C |
| Description | DC 200 fluid | DC 556 fluid | DC 345 fluid |
| | containing | containing | containing |
| | silicone | silicone | silicone |
| | vesicles | vesicles | vesicles |
| Active type | 200 fluid, | 556 fluid | 345 fluid |
| | 10 cSt | | |
| Wt % Loading | 19.8% | 15.9% | 17.3% |
| Starting vesicle | 1A | 1A | 1A |
| sample | | | |
| SPE type for vesicle | Rake SPE | rake SPE | rake SPE |
| Wt. % SPE in vesicle | 20.0% | 20.0% | 20.0% |
| dispersion | | | |
| Composition | | | |
| Si vesicles, g | 75.01 | 75.04 | 60.37 |
| Active amount, g | 3.71 | 2.83 | 2.53 |
| Batch total, g | 78.72 | 77.87 | 62.90 |
| Process condition: | 2 Passes thru | 2 Passes thru | 2 Passes thru |
| | Microfluidizer | Microfluidizer | Microfluidizer |
| | @10,000 psi | @10,000 psi | @10,000 psi |
| Appearance | Translucent | Translucent | Translucent |
| | dispersion | dispersion | dispersion |
| Mv avg. size, μm | 0.082 | 0.086 | 0.069 |
| D(v, 0.5), μm | 0.074 | 0.077 | 0.067 |
| D(v, 0.9), μm | 0.125 | 0.138 | 0.096 |

The Microfluidizer® used was model M-110Y high pressure pneumatic unit, manufactured by Microfluidics Corporation (Newton, Massachusetts). M-110Y Microfluidizer® is a fixed-geometry fluid processor that delivers high shear by forcing the media at high pressure (range from 3,000 to 23,000 psi) through an interaction chamber containing a narrow channel that generates the high shear rate

The silicone fluid loaded silicone vesicles have particle sizes similar to that of the neat vesicles (see example 1, Table 1). The silicone fluid loaded silicone vesicle dispersions in water are homogeneous. No oil separation was observed.

The particle size distributions of the silicone fluid loaded vesicles were plotted against the neat vesicle. No significant difference in particle size profile was observed. This suggests that silicone fluid emollient “filled” in the free volume within the bilayer space of silicone vesicles without causing significant “swelling” or change in the vesicle particle size.

The Cryo-TEM image of silicone fluid loaded silicone vesicles was also obtained, see FIG. 1. Fine silicone vesicles with a mixture of uni-lamellar single vesicle and multi-layered vesicles were found in the DC556 fluid encapsulated silicone vesicles of Example 2B.

The Cryo-TEM image of DC 345 silicone fluid loaded silicone vesicles of was also obtained, as shown in FIG. 2. As seen, fine silicone vesicles with a mixture of uni-lamellar single vesicle and multi-layered vesicles are found in these encapsulated silicone vesicles of Example 2C.

### Example 3

### Preparation of Silicone Actives Containing Silicone Vesicles

Silicone fluid at higher payload levels may also be encapsulated into silicone vesicles following the post-load/shear method. Illustrated in the Table 4 are the examples for DC 556 phenyl(trimethylsiloxyl) siloxane fluid in Example 1A silicone vesicles in water,. prepared from the rake SPE. The starting silicone vesicle dispersion had an average size of 0.150 μm, as shown in the previous section. DC 556 at about 45 wt % payload was successfully prepared to give loaded silicone vesicles in water dispersion of about 0.10 μm size in average The average size of the load silicone vesicles was smaller than the starting neat silicone vesicles, most likely due to the benefit of high shear as the vesicles were processed through the Microfluidizer®.

TABLE 4
DC556 phenyl fluid containing silicone vesicles

| | Example # | | |
|---|---|---|---|
| | 3A | 3B | 3C |
| Description | DC 556 fluid | DC 556 fluid | DC 556 fluid |
| | containing | containing | containing |
| | silicone | silicone | silicone |
| | vesicles | vesicles | vesicles |
| Active type | 556 Fluid | 556 Fluid | 556 Fluid |
| % Loading | 25.38% | 35.25% | 45.24% |
| Vesicle Example # | 1B | 1B | 1B |
| SPE type | rake SPE | rake SPE | rake SPE |
| Wt % SPE | 19.50% | 19.50% | 19.50% |
| Composition | | | |
| Vesicles, g | 70.07 | 70.00 | 70.02 |
| Active, g | 4.65 | 7.43 | 11.28 |
| Batch, g | 74.72 | 77.43 | 81.30 |
| Composition in wt % | | | |
| Wt % SPE vesicles | 18.3% | 17.6% | 16.8% |
| Wt % Actives | 6.2% | 9.6% | 13.9% |
| Wt % Water | 75.5% | 72.8% | 69.3% |
| Processing PSI | 2 passes @ | 2 passes @ | 2 passes @ |
| | 10,000 psi | 10,000 psi | 10,000 psi |
| Appearance | Hazy | Hazy | Hazy |
| | dispersion | dispersion | dispersion |
| Mv avg. size, μm | 0.106 | 0.117 | 0.100 |
| D(V, 0.5), μm | 0.098 | 0.115 | 0.085 |
| D(v, 0.9), μm | 0.167 | 0.167 | 0.171 |

Silicone fluids at even higher payload levels were prepared, as summarized in Table 5 are the examples of DC 200 fluid, 20 cSt encapsulated to about 60 wt % payload in Example 1B silicone vesicles in water, prepared from rake SPE. The starting neat silicone vesicle (1B) contained 19.5 wt % of the SPE and had an average size of 0.150 μm. The resulting DC 200 fluid loaded silicone vesicles were homogeneous dispersions with average size around 0.11 μm.

TABLE 5
Silicone vesicles containing DC 200 fluid at high payload

| | Example # | | |
|---|---|---|---|
| | 3D | 3E | 3F |
| Description | DC 200 fluid | DC 200 fluid | DC 200 fluid |
| | containing | containing | containing |
| | silicone | silicone | silicone |
| | vesicles | vesicles | vesicles |
| Active type | 200 Fluid, | 200 Fluid, | 200 Fluid, |
| | 20 cSt | 20 cSt | 20 cSt |
| Wt % Loading | 30.1% | 45.2% | 60.6% |
| Neat vesicle | 1B | 1B | 1B |
| example # | | | |
| SPE type | rake SPE | rake SPE | rake SPE |
| % SPE in dispersion | 19.50% | 19.50% | 19.50% |
| Formulation amount | | | |
| Vesicles sample, g | 70.00 | 70.06 | 70.10 |
| Active amount, g | 5.889 | 11.25 | 21.04 |
| Batch total, g | 75.89 | 81.31 | 91.14 |
| Composition in % | | | |
| Wt % SPE vesicle | 18.0% | 16.8% | 15.0% |
| Wt % Actives | 7.8% | 13.8% | 23.1% |
| Wt. % Water | 74.3% | 69.4% | 61.9% |
| Processing | 2 passes @ | 2 passes @ | 2 passes @ |
| condition | 10,000 psi | 10,000 psi | 10,000 psi |
| Appearance | Hazy | Hazy | Hazy |
| | dispersion | dispersion | dispersion |
| Mv Avg size, μm | 0.104 | 0.110 | 0.113 |
| D(v, 0.5), μm | 0.086 | 0.098 | 0.095 |
| D(v, 0.9), μm | 0.184 | 0.177 | 0.192 |

### Example 4 (Reference)

### Preparation of Neat Silicone Vesicles from (AB)n SPE

Silicone vesicles in water were prepared from an (AB)n type silicone polyether block copolymer according to the methods of WO2005/103118. A batch of neat silicone vesicles in water was prepared was prepared from a (AB)n SPE of about 50 dp siloxane and Polyglycol AA1200 diallyl polyether. The starting silicone vesicles had an average size about 0.450 μm.

When subjecting the (AB)n SPE type silicone vesicles to high shear, the processed vesicles in water dispersion remained stable and had a smaller size as summarized in Table 6. The post-sheared dispersed vesicles had an average size of 0.179 μm, reduced from 0.450 μm.

TABLE 6
Neat silicone vesicles derived from (AB)n SPE

| | Example # | |
|---|---|---|
| | 4A | 4B |
| | (as made) | Post-sheared |
| SPE type | (AB)n SPE | (AB)n SPE |
| Wt % SPE in dispersion | 19.71% | 19.71% |
| Appearance | Uniform milky | Uniform milky |
| | dispersion | dispersion |
| Process history | As made | 2 passes @ 10,000 psi |
| Mv Avg size, μm | 0.450 | 0.179 |
| D(v, 0.5), μm | 0.395 | 0.174 |
| D(v, 0.9), μm | 0.920 | 0.241 |

### Example 5

### Encapsulating Silicone Fluids into Silicone Vesicles Derived from (AB)n SPE

DC 200 fluid, 20 cSt silicone fluid was loaded into the aqueous (AB)n SPE vesicles of Example 4, as summarized in Table 7. The mixture of the silicone vesicles and silicone fluid was passed through the Microfluidizer twice at a pressure of 10,000 psi. The silicone fluid was successfully incorporated into the silicone vesicles, as evidenced by the homogeneous appearance which also remained as a water-continuous dispersion. The loaded silicone vesicles had an average size smaller than that of the un-processed vesicles (0.45 μm for Example 4A) and somewhat larger than that of the unloaded, processed vesicles (0.179 μm for the processed Example 4B).

TABLE 7
DC 200 fluid containing silicone vesicles from (AB)n SPE

| | Example # | | | |
|---|---|---|---|---|
| | 5A | 5B | 5C | 5D |
| Active type | 200 Fluid, 20 cSt | 200 Fluid, 20 cSt | 200 Fluid, 20 cSt | 200 Fluid, 20 cSt |
| Wt % loading | 29.6% | 35.2% | 40.0% | 44.9% |
| Vesicle | 4A | 4A | 4A | 4A |
| Example # | | | | |
| SPE type in | (AB)n SPE | (AB)n SPE | (AB)n SPE | (AB)n SPE |
| vesicles | | | | |
| Wt % SPE in | 19.71% | 19.71% | 19.71% | 19.71% |
| vesicle | | | | |
| Amt vesicles, g | 70.03 | 70.04 | 70.07 | 70.06 |
| Amt active, g | 5.806 | 7.502 | 9.197 | 11.251 |
| Batch total, g | 75.8 | 77.5 | 79.3 | 81.3 |
| Appearance | Milky | Milky | Milky | Milky |
| | dispersion | dispersion | dispersion | dispersion |
| Process history | 2 passes @ | 2 passes @ | 2 passes @ | 2 passes @ |
| | 10,000 psi | 10,000 psi | 10,000 psi | 10,000 psi |
| Mv avg size μm | 0.275 | 0.300 | 0.289 | 0.338 |
| D(v, 0.5), μm | 0.264 | 0.264 | 0.273 | 0.238 |
| D(v, 0.9), μm | 0.395 | 0.497 | 0.445 | 0.766 |

### Example 6

### Encapsulating Fragrance into Silicone Vesicles

Fragrance, perfume oil, or flavors compounds may also be incorporated into silicone vesicles to form a stable dispersion in water, following the current method. Illustrated in the Table 8 are examples of fragrance loaded silicone vesicles in water. The starting neat silicone vesicle used in these examples was Example 6A, a neat silicone vesicle similar to Example 1A, except it was prepared from an AE501 monoallyl polyether derived rake SPE of MD₉₄D^{(EO12)}₆M structure.

These examples were prepared according to the following procedure:
1. Incorporate fragrance into silicone vesicles in water dispersion,
2. Mix or shake to disperse fragrance using mechanical stirrer, shaker or vibrator,
3. Subject the above mixture to Microfluidizer® or equivalent high-shear device at a pre-determined pressure settings (17,000 psi in the case of Microfluidizer),
4. Return the effluent of Microfluidizer processed mixture to one more pass, to give a total of 2 passes through the Microfluidizer
5. Inspect the appearance and the particle size distribution of the final mixture.

The composition and properties of fragrance containing silicone vesicle in water dispersion are summarized in Table 8. The wt % payload is the amount of fragrance divided by the total of silicone vesicles and fragrance.

TABLE 8
Fragrance loaded silicone vesicles

| | Example # | | |
|---|---|---|---|
| | 6A | 6B | 6C |
| Description | Neat silicone | Fragrance | Fragrance |
| | vesicles from | loaded silicone | loaded silicone |
| | the rake SPE | vesicles | vesicles |
| Active type | None | Fragrance A | Fragrance B |
| Wt % active load | 0 | 11.3 | 11.6 |
| Process description | As made | 2 Passes @ | 2 Passes @ |
| (Microfluidizer ®) | | 17,000 psi | 17,000 psi |
| Starting neat silicone | None | 6A | 6A |
| vesicle batch | | | |
| Starting vesicle | 100.0 | 101.4 | 75.3 |
| composition, g | | | |
| Fragrance, g | 0.0 | 2.5 | 1.9 |
| Batch total, g | 100.0 | 103.9 | 77.2 |
| Wt % vesicle solids | 19.4 | 18.9 | 18.9 |
| Wt % fragrance | 9.0 | 2.4 | 2.5 |
| Wt % water phase | 80.6 | 78.7 | 78.6 |
| Appearance | Translucent | Translucent | Slightly hazy, |
| | clear, | clear, | homogeneous |
| | homogeneous | homogeneous | |
| Dispersion particle | | | |
| Mv avg. size, μm | 0.098 | 0.071 | 0.085 |
| D(v, 0.5), μm | 0.092 | 0.066 | 0.079 |
| D(v, 0.9), μm | 0.148 | 0.104 | 0.127 |

Additional examples of fragrance loaded silicone vesicles were prepared using the silicone vesicle dispersion as prepared from the (AB)n SPE. The (AB)n SPE was the hydrosilylation product of dimethylsiloxyl-terminated PDMS of 50 dp and αω-diallyl-terminated poly(oxyethylene) glycol. The composition and property of fragrance loaded silicone vesicle dispersion is shown in Table 9.

TABLE 9
Fragrance loaded silicone vesicles derived from (AB)n SPE

| | Example # | |
|---|---|---|
| | 6D | 6E |
| Description | 10% Neat vesicles | Fragrance loaded silicone |
| | from the (AB)n SPE | vesicles from the (AB)n |
| | | SPE |
| Actives | None | Fragrance |
| Wt % active load | 0.0 | 11.3 |
| Process description | As made | 2 Passes thru Microfluidizer |
| | | @ 17,000 psi |
| Starting neat silicone | none | 6D |
| vesicle ID | | |
| Silicone vesicle, g | 100.0 | 82.2 |
| Fragrance, g | 0.0 | 1.1 |
| Batch total, g | 100.0 | 83.3 |
| Wt % vesicle solids | 10.2 | 10.0 |
| Wt % active | 0.0 | 1.3 |
| Wt % water | 89.8 | 88.7 |
| Appearance | Homogeneous milky | Homogeneous milky |
| Particle size | | |
| Mv avg. size, μm | 0.248 | 0.107 |
| D(v, 0.5), μm | 0.082 | 0.090 |
| D(v, 0.9), μm | 1.072 | 0.180 |

### Example 7

### Encapsulating Fragrance and Silicone Fluid into Silicone Vesicles

In many instances, it is desirable to use a silicone fluid emollient to form a uniform mixture of fragrance and silicone fluid. The fragrance/silicone fluid mixture can then be loaded into silicone vesicles to form a homogeneous dispersion in water.

This example used the following procedure:
1. Prepare a homogeneous mixture of fragrance and a silicone fluid of choice,
2. Incorporate the fragrance/silicone fluid mixture into silicone vesicles in water dispersion,
3. Mix or shake to disperse fragrance/silicone fluid using mechanical stirrer, shaker or vibrator,
4. Subject the above mixture to Microfluidizer® or equivalent high-shear device at a pre-determined pressure settings (17,000 psi in the case of Microfluidizer),
5. Return the effluent of Microfluidizer processed mixture to one more pass, to give a total of 2 passes through the Microfluidizer
6. Inspect the appearance and the particle size distribution of the final mixture.

The composition and properties of the fragrance/silicone fluid containing silicone vesicle in water dispersion prepared in this example are shown in Table 10. The wt % payload is the amount of fragrance divided by the total of silicone vesicles and fragrance.

TABLE 10
Fragrance/silicone fluid loaded silicone vesicles from rake SPE

| | Example # | |
|---|---|---|
| | 7A | 7B |
| Description | 10% Neat vesicles | Fragrance/556 fluid loaded |
| | from the rake SPE | silicone vesicles |
| Active type | None | Fragrance/556 Fluid |
| Wt % active load | 0.0 | 10.5 |
| Process description | As made | 2 Passes thru Microfluidizer |
| | | @ 17,000 psi |
| silicone vesicle ID | None | 7A |
| Starting vesicle, g | 100.0 | 102.6 |
| Fragrance active, g | 0.0 | 1.3 |
| Silicone fluid, g | 0.0 | 1.3 |
| Batch total, g | 100.0 | 105.1 |
| Wt % vesicle solids | 10.5 | 10.2 |
| Wt % active | 0.0 | 1.2 |
| Wt % silicone fluid | 0.0 | 1.2 |
| Wt % water phase | 89.6 | 87.5 |
| Appearance | Translucent clear, | Homogeneous milky |
| | homogeneous | dispersion |
| Mixture property | | |
| Mv avg. size, μm | 0.063 | 0.072 |
| D(v, 0.5), μm | 0.060 | 0.069 |
| D(v, 0.9), μm | 0.088 | 0.099 |

### Example 8

### Encapsulating Vitamin into Silicone Vesicles

Vitamin A Palmitate (VAP) was incorporated into a silicone vesicle dispersion as summarized in Table 11. The neat silicone vesicles in Example 6A was used in this examples. In the first set of examples VAP was loaded directly into Example 6A silicone vesicles to give VAP loaded vesicles; in the second set of examples, a mixture of VAP and DC 1-2287 silicone fluid was formed, then incorporated into the silicone vesicles.

The composition and the properties of fragrance containing silicone vesicle in water dispersion are shown in the Table 11. The wt % active and silicone fluid payloads are shown. The VAP used in this invention contains about 1.5 wt % butylated hydroxytoluene (BHT) stabilizer.

TABLE 11
Vitamin A palmitate loaded silicone vesicles

| | Example # Sample ID | | | |
|---|---|---|---|---|
| | 8A | 8B | 8C | 8D |
| Description | Neat vesicles | VAP loaded | Neat vesicles | VAP/1-2287 |
| | from rake SPE | vesicles | from rake SPE | loaded vesicles |
| Active type | None | VAP | None | VAP/1-2287 |
| Wt % active load | 0 | 12.0 | 0.0 | 13.7 |
| Process | As made | 2 Passes thru | As made | 2 Passes thru |
| description | | @ 17,000 psi | | @ 17,000 psi |
| Starting neat | | 6A | | 6A |
| vesicle ID | | | | |
| Starting vesicle | 100.0 | 75.2 | 100.0 | 104.3 |
| composition, g | | | | |
| Vitamin A | 0.0 | 2.0 | 0.0 | 1.7 |
| palmitate, g | | | | |
| Silicone fluid, g | 0.0 | 0.0 | 0.0 | 1.9 |
| Batch total, g | 100.0 | 77.1 | 100.0 | 108.0 |
| Wt % vesicle | 19.4 | 18.9 | 10.5 | 10.1 |
| solids | | | | |
| Wt % VAP | 0.0 | 2.6 | 0.0 | 1.6 |
| Wt % silicone | 0.0 | 0.0 | 0.0 | 1.8 |
| fluid | | | | |
| Wt % water | 80.6 | 78.5 | 89.6 | 86.6 |
| phase | | | | |
| Appearance | Translucent, | Homogeneous milky | Translucent, | Slightly hazy, |
| | homogeneous | | homogeneous | dispersion |
| Mixture property | | | | |
| Mv avg. size, μm | 0.098 | 0.174 | 0.063 | 0.078 |
| D(v, 0.5), μm | 0.092 | 0.113 | 0.060 | 0.069 |
| D(v, 0.9), μm | 0.148 | 0.410 | 0.088 | 0.121 |

1. A process for preparing a hydrophobic active loaded vesicle composition comprising:
I) combining;
A) an organopolysiloxane having at least one hydrophilic substituent group,
B) a water miscible volatile solvent,
C) optionally, a silicone or organic oil, with water to form an aqueous dispersion,
II) mixing the aqueous dispersion to form a vesicle dispersion,
III) optionally, removing the water miscible volatile solvent from the vesicle dispersion, and then
IV) admixing to the vesicle dispersion;
D) a hydrophobic active to form the hydrophobic active loaded vesicle composition.

2. The process of claim 1 wherein the organopolysiloxane is a silicone polyether having the formula: 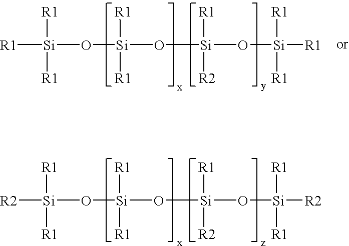
where R1 represents an alkyl group containing 1-6 carbon atoms;
R2 represents the group —(CH₂)ₐO(C₂H₄O)_{b}(C₃H₆O)_{c}R3;
x is 1-1,000; y is 1-500; z is 1-500; a is 3-6; b is 4-20; c is 0-5:
and R3 is hydrogen, a methyl group, or an acyl group.

3. The process of claim 1 wherein the organopolysiloxane is a (AB)ₙ block silicone polyether having the formula:
—[R¹(RₛSiO)_{x′}(R₂SiR¹O)(CₘH₂ₘO)_{y}]ₙ— [Formula 1]
where x′ and y′ are greater than 4, m is from 2 to 4 inclusive. n is greater than 2.
R is independently a monovalent organic group containing 1 to 20 carbons,
R¹ is a divalent hydrocarbon containing 2 to 30 carbons.

4. The process of claim 1 wherein the water miscible volatile solvent is an alcohol.

5. The process of claim 4 wherein the alcohol is ethanol or isopropanol.

6. The process of claim 1 wherein component C) is present and is a volatile methyl siloxane.

7. The process of claim 1 wherein the hydrophobic active is selected from the group,
D′) a silicone oil,
D″) a personal care active,
D′″) a healthcare active,
and mixtures thereof.

8. The process of claim 7 wherein the silicone oil is a polydimethylsiloxane.

9. The process of claim 7 wherein the silicone oil is a phenyl functional organopolysiloxane.

10. The process of claim 7 wherein the silicone oil is an organocyclosiloxane.

11. The process of claim 7 wherein the hydrophobic active is a hydrophobic vitamin.

12. The process of claim 7 wherein the personal care active is a sunscreen agent.

13. The process of claim 7 wherein the personal care active is a fragrance or perfume.

14. The process of claim 1 wherein the healthcare active is a pharmaceutical drug.

15. The process of claim 1 wherein the admixing in step IV) comprises a shear nixing process.

16. A vesicle composition prepared according to the process of claim 1.

17. A personal care product comprising the vesicle composition of claim 16.

18. The personal care product of claim 17 wherein the personal care product is selected from an antiperspirant, deodorant, skin cream, skin care lotion, moisturize, facial treatment, wrinkle remover, facial cleansers, bath oils, sunscreens, pre-shave, after-shave lotions, liquid soap, shaving soap, shaving lather, hair shampoo, hair conditioner, hair spray, mousse, permanent, hair cuticle coat, make-up, color cosmetic, foundation, blush, lipstick, lip balm, eyeliner, mascara, nail polishes, and powders.
